(19) United States
(12) Patent Application Publication
(10) Pub. No.: US 2005/0107700 A1
(43) Pub. Date: May 19, 2005

(54) Thin film ultrasonic transmitter

(76) Inventors: Steven Taylor Morris, Madison, WI (US); Richard Franklin Morris, Stoughton, WI (US)

Correspondence Address: QUARLES & BRADY LLP, 411 E. WISCONSIN AVENUE, SUITE 2040, MILWAUKEE, WI, 53202-4497, US

(21) Appl. No.: 10/713,417

(22) Filed: Nov. 14, 2003

(51) Int. Cl. A61B 8/00

(52) U.S. Cl. 600/437; 600/458

(57) ABSTRACT

A thin film piezoelectric material employs a metallic backer plate to provide high output, non-resonant ultrasonic transmissions suitable for quantitative ultrasonic measurement and/or imaging.

### CROSS-REFERENCE TO RELATED APPLICATIONS

--

### STATEMENT REGARDING FEDERALLY SPONSORED RESEARCH OR DEVELOPMENT

--

### BACKGROUND OF THE INVENTION

The present invention relates to ultrasonic acoustic transmitters, such as may be used in transmission quantitative ultrasonic imaging and measurements, and in particular to an improved thin film transmitter.

Transmitting ultrasonic transducers are used, for example, in bone densitometers where ultrasound is transmitted through in vivo bone, most typically the os calcis of the heel, in order to measure trabecular bone. Common measurements made by such densitometers include the speed of sound (SOS) and broadband ultrasonic attenuation (BUA) in the bone. Images of the bone based on these or other measurements may also be provided by the densitometer. Densitometers of this type are described in U.S. Pat. Nos. 5,840,029 and 6,517,487 assigned to the assignee of the present invention and hereby incorporated by reference.

Ceramic transducers are commonly used as the transmitting ultrasonic transducer in such densitometers because of their high output signals. In this application, the mechanical resonance of the ceramic transducer is adjusted to be near the principal frequency being transmitted. Operation in this “resonant” mode increases the output of the transducer, but can make manufacturing of the transducer difficult because of the high sensitivity of the transducers resonant frequency to variations in the dimensions of the many subcomponents of the transducer. Slight differences in resonant frequencies of the transducers on different machines complicate the effort to provide highly repeatable measurements that are machine independent.

Thin film polymer piezoelectric materials such as polyvinylidene fluoride (PDVF) may also be used as an ultrasonic transducer as described in U.S. Pat. No. 6,305,060 issued Oct. 23, 2001 and U.S. Pat. No. 6,012,779 issued Jan. 11, 2000 assigned to the assignee of the present invention and hereby incorporated by reference. Application of PVDF to transmitting ultrasonic transducers has been limited because of low output levels.

### SUMMARY OF THE INVENTION

The present invention provides an ultrasonic transmitter using a piezoelectric film and suitable for use in transmission quantitative ultrasonic imaging systems. The transducer provides suitable output levels and may operate in a non-resonant mode avoiding some of the difficulties of manufacturing present ceramic transducers.

Generally, the invention employs a thin metallic backer plate to the piezoelectric film that provides a sharp discontinuity in acoustic impedance at the back surface of the piezoelectric film that increases the acoustic output from the piezoelectric film's front surface. The low acoustic attenuation of the metal of the metallic plate, which would normally cause reflections off of the metal plate's back surface to interfere with the wave in the piezoelectric film, is minimized by adjusting the plate thickness to provide a proper phasing of reflections that reduces this interference. The transducer may be essentially non-resonant to reduce coloration of the signal, and the metallic plate may provide an electrode for the piezoelectric film, further simplifying the manufacturing process.

### BRIEF DESCRIPTION OF THE FIGURES

FIG. 1 is a perspective exploded view of the ultrasonic transducer of the present invention showing a protective acoustically transparent layer followed by a thin film piezoelectric material, a metallic backer plate and support structure;

FIG. 2 is a fragmentary, elevational cross section through the transducer of FIG. 1 showing the layers of the transducer as assembled and the connection of electrodes to opposite sides of the piezoelectric material; and

FIG. 3 is a simplified diagram of a quantitative ultrasonic densitometer using the transducer of the present invention.

### DETAILED DESCRIPTION OF THE PREFERRED EMBODIMENT

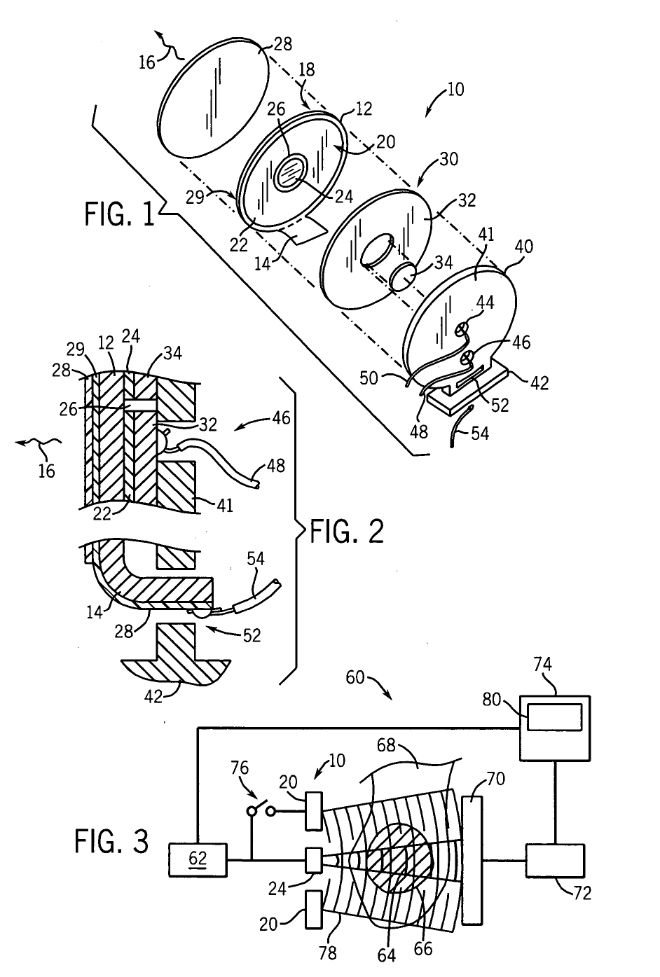

Referring now to FIG. 1, an ultrasonic transmitting transducer 10 constructed according to the present invention includes a disk-shaped piezoelectric film 12.

In the preferred embodiment, the piezoelectric film 12 is a polyvinylidene fluoride film (PVDF) that has been polarized to create piezoelectric properties according to methods well understood in the art. The piezoelectric film 12 includes a tab 14 extending downwardly from an edge of the disk which may be folded rearward as shown.

A front face 18 of the piezoelectric film 12 and the front side of the tab 14 may be coated with a conductive material such as copper by vacuum metallization or electroplating to create a front electrode 29. This front electrode 29 may be coated with nickel to reduce corrosion and will provide an essentially continuous conductive surface over the front face 18, and lower surface of the tab 14 when the tab 14 is folded backward as shown in FIG. 1.

The front face 18 of the piezoelectric film 12 and the front electrode 29 may then be covered by an acoustically transparent protective film 28 such as Teflon to prevent direct contact between water or other acoustic coupling medium and the front electrode 29.

A rear face 20 of the piezoelectric film 12 may be similarly coated with conductive material to form an outer annular electrode 22 and an inner disk-shaped electrode 24 centered within the outer annular electrode 22 and electrically isolated from the outer annular electrode 22 by a small ring-shaped gap 26.

Attached to the rear face 20 of the piezoelectric film 12 is a metallic backer plate assembly 30 having an outer annular plate 32 of area substantially equal to that of outer annular electrode 22 and a center disk plate 34 corresponding in area to and aligning with the inner disk-shaped electrode 24. The alignment assures that the center disk plate 34 only touches inner disk-shaped electrode 24 and not outer annular electrode 22 and outer annular plate 32 touches only outer annular electrode 22 and not inner disk-shaped electrode 24. Bonding between the backer plate assembly 30 and the piezoelectric film 12 may be done through the use of a thin adhesive such as epoxy, a process simplified by the flexible nature of the piezoelectric film 12. The outer annular electrode 22 and inner disk-shaped electrode 24 are optional and may be replaced simply with the metallic surfaces of the backer plate assembly 30 such that the charge pushed to the PVDF is transferred via the close proximity of the metal backing plate.

The outer annular electrode 22 and a center disk plate 34 of the metallic backer plate assembly 30 is preferably 0.025 inch stainless steel. The metal of the backer plate assembly 30 has an acoustic impedance substantially different from the material of the piezoelectric film 12 to reduce but not eliminate acoustic coupling between the two. In an alternative embodiment, the outer annular electrode 22 and a center disk plate 34 of the metallic backer plate assembly 30 may be composed of many electrically independent electrodes covering generally the regions of the outer annular electrode 22 and a center disk plate 34, for phased array operation.

The piezoelectric film 12 and the backer plate assembly 30 may all be held within an injection molded housing 40 having a mounting base 42 supporting a disk-shaped support plate 41. The disk-shaped support plate 41 may abut a rear face of the backer plate assembly 30 to support the same while exposing the front face 18 of the piezoelectric film 12 under the protective film 28. The injection molded housing 40 may be constructed of a thermoplastic having an acoustic impedance differing substantially from the metal of the backer plate assembly 30 minimizing acoustic transmission through this interface as will be understood to those of ordinary skill in the art.

The disk-shaped support plate 41 has two holes 44 and 46 through its surface and opening at its rear surface to allow electrode leads 50 and 48 to pass through the housing 40 to electrically contact center disk plate 34 and outer annular plate 32, respectively. Center disk plate 34 and outer annular plate 32 may capacitively couple to the outer annular electrode 22 and inner disk-shaped electrode 24, respectively, or may be joined to the outer annular electrode 22 and inner disk-shaped electrode 24, respectively, using a conductive paste or epoxy or the like.

When the piezoelectric film 12 and backer plate assembly 30 is mounted on the housing 40, the tab 14 of the piezoelectric film 12 passes through a slot 52 between the disk-shaped support plate 41 and the mounting base 42 to permit the attachment of electrode lead 54 to the tab 14 at the rear of the housing 40.

When energized, the piezoelectric film 12 will create an acoustic signal 16 directed generally along a longitudinal axis perpendicular to a front face 18 of the piezoelectric film 12. While the inventor does not wish to be bound by a particular theory, it is believed that a similar signal will be generated in the opposite direction to be reflected by the backer plate assembly 30 as a result of the difference of acoustic properties of the PDVF and stainless steel. The small amount of acoustic energy conducted coupled into the stainless steel of the backer plate assembly 30 is largely reflected at the interface between the backer plate assembly 30 and the housing 40, and the thickness of the backer plate assembly 30 is adjusted so that the phase of the returning signal (part of which will be coupled into the piezoelectric film 12), adds constructively to the existing signal.

The resulting transducer is essentially non-resonant at ultrasonic frequencies (as defined both by center frequency and Q) and has lower construction costs than a ceramic device. Because of the low resonance of the ultrasonic transmitting transducer 10, the output wave is not colored by the resonant characteristics providing improved device-to-device consistency. Although the present inventors do not wish to be bound by a particular theory, they believe that the thin film piezoelectric film 12 has an additional advantage over ceramic as a transmitter in that it provides very little lateral mode wave such as improves the beam profile produced by ultrasonic transmitting transducer 10 in the region of the outer annular plate 32. The resulting ultrasonic transmitting transducer 10 can have an operating bandwidth of 3 MHz or more, compared to a 300 KHz bandwidth achievable with ceramic transducers.

Referring now to FIG. 3, an ultrasonic densitometer 60 employing the ultrasonic transmitting transducer 10 of the present invention may provide an oscillator 62 operating in two modes, one for quantitative ultrasound and one for imaging. In a quantitative ultrasound mode, the oscillator 62 may provide a 500 kilohertz pulse energizing only the central section of the piezoelectric film 12 associated with the inner disk-shaped electrode 24 to produce a relatively narrow beam 64 through the os calcis 66 of the human heel 68. That narrow beam 64 may be detected by a receiving transducer 70 providing a signal amplified by an amplifier 72 and passed to a processing computer 74 for quantitative measurements.

In imaging mode, the oscillator 62 may be operated at a substantially higher frequency, for example, one megahertz and both the piezoelectric film 12 associated with inner disk-shaped electrode 24 and associated with outer annular electrode 22 may be energized through switch 76 to provide a broad area beam 78 to be detected by the receiving transducer 70 to provide an image that may be generated on a display screen 80 of the computer 74.

It is specifically intended that the present invention not be limited to the embodiments and illustrations contained herein, but include modified forms of those embodiments including portions of the embodiments and combinations of elements of different embodiments as come within the scope of the following claims. For example, although the invention has been described in the context of a transmitter only, it will be appreciated that it can be used as part of a transceiver that provides for transmission and reception of ultrasonic signals. Accordingly, the word transmitter in the claims should not operate to preclude use as both a transmitter and receiver.

1. An ultrasonic transmitter comprising:
a piezoelectric polymer film adapted to transmit an ultrasonic acoustic signal from a front face along a signal path; and
a metallic backer plate adhered to a rear face of the piezoelectric polymer film having a thickness along the signal path substantially thinner than one wavelength.

2. The ultrasonic transmitter of claim 1 further including a support structure supporting the metallic backer plate and having an acoustic impedance substantially different from the metallic backer plate.

3. The ultrasonic transmitter of claim 1 wherein the support structure is a thermoplastic material.

4. The ultrasonic transmitter of claim 1 wherein the metallic backer plate includes an outer annular portion surrounding an independently supported inner center portion.

5. The ultrasonic transmitter of claim 4 further including a first lead attached to a flexible conductor on the front face of the piezoelectric polymer film to provide a first electrode and second and third leads attached to the outer annular portion and the inner center portion, respectively, to provide independent secondary electrodes.

6. The ultrasonic transmitter of claim 5 further including an outer annular electrode attached on a second face of the piezoelectric polymer film aligned with the surrounding outer annular portion of the backer plate and an inner center portion electrode aligned with the inner center portion of the backer plate.

7. The ultrasonic transmitter of claim 1 wherein the piezoelectric polymer film is disk-shaped.

8. The ultrasonic transmitter of claim 1 further including an oscillator attached to the piezoelectric polymer film operating at a frequency removed from the natural resonance of the ultrasonic transducer.

9. The ultrasonic transmitter of claim 1 wherein the metallic backer plate is stainless steel.

10. The ultrasonic transmitter of claim 1 wherein the backer plate is substantially less than ½ wavelength of the frequency of the acoustic signal.

11. The ultrasonic transmitter of claim 1 wherein the metallic backer plate is less than 0.05 inches thick.

12. The ultrasonic transmitter of claim 1 wherein the backer plate is substantially 0.025-inch thick stainless steel.

13. The ultrasonic transmitter of claim 1 wherein the piezoelectric polymer film is PVDF.

14. The ultrasonic transmitter of claim 1 wherein the metallic backer plate is composed of a plurality of independent electrodes.

15. A quantitative imaging ultrasound device comprising:
an ultrasonic transducer having a piezoelectric polymer film adapted to transmit an ultrasonic acoustic signal from a front face along a signal path and a metallic backer plate adhered to a rear face of the piezoelectric polymer film;
an ultrasonic receiver for receiving the ultrasonic acoustic signal along a signal path through a portion of the human body; and
an output device providing an image formed from the received ultrasonic acoustic signal.

16. The imaging ultrasound device of claim 15 further including a support structure supporting the metallic backer plate and having an acoustic impedance substantially different from the metallic backer plate.

17. The imaging ultrasound device of claim 15 wherein the support structure is a thermoplastic material.

18. The imaging ultrasound device of claim 15 wherein the piezoelectric polymer film provides separate electrical connections to an outer annular portion surrounding an independently supported inner center portion.

19. The imaging ultrasound device of claim 18 further including an oscillator attached to the electrodes of the piezoelectric polymer film to provide different signals to the inner center portion and the outer annular portion for quantitative measurements and imaging.

20. The imaging ultrasound device of claim 19 wherein the different frequencies are removed from modes of natural resonance of the ultrasonic transducer.

21. The imaging ultrasound device of claim 20 wherein the backer plate has a thickness substantially equal to an integer multiple of one-half the wavelength of the principle frequency of the oscillator as measured in the metallic backer plate.

22. The imaging ultrasound device of claim 15 wherein the metallic backer plate is composed of a plurality of independent electrodes.
